(19) United States
(12) Patent Application Publication
(10) Pub. No.: US 2020/0297217 A1
(43) Pub. Date: Sep. 24, 2020

(54) INTRAVASCULAR MONITOR

(71) Applicant: SHIFAMED HOLDINGS, LLC, Campbell, CA (US)

(72) Inventor: Tom SAUL, Moss Beach, CA (US)

(73) Assignee: SHIFAMED HOLDINGS, LLC, Campbell, CA (US)

(21) Appl. No.: 16/087,770

(22) PCT Filed: Mar. 27, 2017
(86) PCT No.: PCT/US2017/024244

§ 371 Date: Sep. 24, 2018

Related U.S. Application Data
(60) Provisional application No. 62/313,669, filed on Mar. 25, 2016.

(51) Int. Cl. A61B 5/0215; A61B 5/00; A61B 5/023; A61B 5/0235

(52) U.S. Cl. CPC A61B 5/0215; A61B 5/6852; A61B 5/74; A61B 5/0235; A61B 5/023

(57) ABSTRACT

Intravascular blood pressure monitoring devices and methods that include an indwelling portion and a pressure transducer. The indwelling portion may include a tubular member, at least a portion of the tubular member configured to transmit fluid pressure from blood that is external to the tubular member to a fluid disposed within the tubular member, and the tubular member may be adapted to isolate the fluid from the blood, with the fluid within the tubular member being in pressure communication with a pressure transducer. The devices may include a stiffening member interface adapted to releasably interface with a stiffening member to stiffen at least a portion of the indwelling portion while delivering the indwelling portion to a position within the subject, and causing the indwelling portion to be less stiff after its removal.

### CROSS REFERENCE TO RELATED APPLICATIONS

This application claims the benefit of U.S. Provisional Application No. 62/313,669, filed Mar. 25, 2016, which is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

FIG. 1A illustrates the primary components of a traditional intravascular blood pressure (BP) monitoring system configured for monitoring arterial pressure. Such a system typically comprises: a cannula typically in the range of 18 to 22 ga for access to the inner lumen of a vessel, typically a radial artery; a pressure transducer in a housing configured as a flow through device; a short piece of non-compliant tubing to connect the cannula to the pressure transducer; a reservoir of sterile fluid (e.g., saline) to constantly flush the transducer and associated lines, a stopcock (valve) to control the flow of the fluid, a pressurizing device (e.g., a pressure bulb) to apply pressure to the fluid reservoir, associated tubing to connect the fluid reservoir to the pressure transducer, and an monitoring instrument adapted to monitor and record the data monitored by the pressure transducers. The fluid is used to flush the transducer and thereby keep blood from clotting within the system, which would negatively impact the pressure monitoring capabilities of the pressure transducer. Such a system also provides a way of accessing arterial blood for the purposes of collecting blood samples when they are required on a relatively frequent basis, such as when blood gas values are required. Such systems are typically limited to use in critical care settings where patients are not or minimally ambulatory. As such, there has been very little drive to modify such systems to accommodate ambulatory patients and to design systems more conducive to use in noncritical care settings.

Presently, patients outside of the critical care setting are monitored by noninvasive intermittent BP monitoring systems, such as is described in U.S. Pat. No. 7,029,448 B2. Such intermittent monitoring often misses critical events which, if detected, would allow for better treatment of the patient. Additionally, since the subject is aware that the reading is taking place due to noise and the tightening of the cuff, the occurrence of the reading often influences the subject enough to cause a change in BP.

There are many patients both in a hospital setting and at home who could benefit from a real-time, continuous, BP monitoring system for use by ambulatory patients.

### SUMMARY OF THE DISCLOSURE

One aspect of the disclosure is an intravascular blood pressure monitoring system, comprising: a patient interface including an indwelling portion that is adapted to be positioned within a blood vessel, the indwelling portion comprising a tubular member, at least a portion of the tubular member configured to transmit fluid pressure from blood that is external to the tubular member to a fluid disposed within the tubular member, the tubular member adapted to isolate the fluid from the blood, the fluid within the tubular member being in pressure communication with a pressure transducer, and a stiffening member interface adapted to releasably interface with a stiffening member to stiffen at least a portion of the indwelling portion while delivering the indwelling portion to a position within the subject, and causing the indwelling portion to be less stiff after its removal.

The pressure transducer can be adapted to be disposed outside of the blood vessel.

The patient interface can include an external portion, and the external portion can be configured to be secured to the skin of a subject.

The outer wall can have a thickness less than 0.25 mm.

The indwelling portion can have a section proximal to the elongate section, wherein a thickness of an outer wall in the proximal section is greater than the thickness of the outer wall in the elongate section. The fluid can be disposed in a lumen through which the stiffening member is configured to be positioned. The system can further include a central elongate member secured to a section of the indwelling portion distal to the elongate section, the central elongate member configured with a surface to interface with the stiffening element.

The indwelling portion can further include a one way valve distal to the elongate section, allowing fluid to pass only distally through the one way valve.

The indwelling portion can include a sharpened distal end.

The patient interface can include a communication component adapted to communicate with an external monitor.

One aspect of the disclosure is an intravascular blood pressure monitoring apparatus, comprising: a patient interface including an indwelling portion that is adapted to be positioned within a blood vessel, the indwelling portion including a pressure transducer disposed in a distal region of the indwelling portion; and a stiffening member interface adapted to releasably interface with a stiffening member to stiffen at least a portion of the indwelling portion while delivering the indwelling portion to a position within the subject, and causing the indwelling portion to be less stiff after its removal.

The pressure transducer can be in electrical communication with a signal conditioner.

The indwelling portion can have a sharpened distal end.

The patient interface can include a communication component adapted to communicate with an external monitor.

The patient interface can comprise an external portion, the external portion configured to be secured to the skin of a subject.

The exemplary Ambulatory RF link enabled Intravascular Monitor (ARLIM) systems described herein overcome one or more of the deficiencies of traditional non-ambulatory systems set forth above.

In some embodiments the ARLIM system includes a patient interface and a monitor for recording and processing information transmitted from the patient interface. Generally, the patient interface will comprise at least an access device and a pressure transducer. A number of variations for each of these components are described in the embodiments detailed herein.

The exemplary pressure transducers described herein may be chosen from any known in the art, which can be configured for use in the dimensions required by the devices, systems, and methods described herein. Such pressure transducers that can be used include piezo resistive and capacitance based devices. In some embodiments described herein, the pressure transducer is located within a distal region of the access device, and in other embodiments it is located at a proximal region (e.g., a proximal end) of the access device. When the pressure transducer is located within a distal region of the access device, the pressure transducer is introduced into the blood stream. In some such embodiments the access device comprises a rigid tip adapted and configured to puncture the tissue, and a compliant portion that spans the distance between the pressure transducer, through the tissue, to a portion of the system interface disposed on the outer surface of the skin. In some embodiments the compliant portion comprises a fluid path open to the blood.

In some embodiments the pressure transducer is located in a proximal region of an access device, and is fluidically coupled to the blood via a fluid path in the access device. In such embodiments, the pressure transducer can be disposed in a portion of the patient interface that resides on the outer surface of skin. The fluidic coupling may couple a fluid such as saline, a gel medium, silicone oil, or some other suitable fluid. The fluid path in some configurations will be sealed to fluid transport, but allow for the transmission of the pressure signal.

A fluid path provided in some access devices described herein can additionally provide access in a proximal region of the fluid path via membranes, valves, or fittings, for example. Such fluid paths are useful for purging the fluid path, and/or injection of fluids into the blood stream.

In some of the embodiments described herein, an introducer device is used to deliver the distal region of the access device into the blood stream. An introducer for use with an access device that comprises a puncturing tip can interface with the proximal region of the puncturing tip.

In some embodiments the patient interface comprises an access device and a signal conditioning component that communicates with the system interface via an RF link.

In some embodiments the patient interface comprises an access device that connects to the system via a connector comprising physical electrical contacts.

In some embodiments the pressure transducer resides in the portion of the patient interface delivered within the vessel being monitored. In some embodiments the pressure transducer resides in the portion of the patient interface external to the vessel.

In some embodiments the ARLIM system comprises an introducer. In some such embodiments the ARLIM system is adapted to store the introducer after use in delivering the access device in a manner that protects the patient from future puncture. In some embodiments the introducer is separate and removable from the system, which lends itself to a removable introducer.

In some embodiments the devices and methods provide real time continuous data recorded on smart phone, other wireless device, or other remote device.

In some embodiments the devices and methods are less obtrusive then traditional systems. They can have a low profile flexible configuration, and a compliant, smooth edged external device that is easily covered with wrap

In some embodiments the devices and methods provide ways of sampling or introducing fluids.

In some embodiments portions of the devices can be fully implanted.

In some embodiments the devices and methods.

In some embodiments the devices and methods are more flexible after removal of a stylet, and are therefore more comfortable.

In some embodiments the devices and methods do not require drip to maintain patency.

In some embodiments the devices and methods are less susceptible to clotting.

In some embodiments the accuracy is less susceptible to errors due to deposits on sensor.

### BRIEF DESCRIPTION OF THE DRAWINGS

FIGS. 1A and 1B illustrate components in an exemplary traditional system, and a block diagram of such a traditional system.

FIG. 2A illustrates an exemplary patient interface and positioning of a sensor.

FIG. 2B illustrates a block diagram of an exemplary system, including the exemplary patient interface from FIG. 2A.

FIG. 3 illustrates an exemplary embodiment of a system, including placement of an intraarterial pressure sensor.

FIG. 4 illustrates an exemplary patient interface, including exemplary position of a pressure transducer.

FIGS. 5A and 5B illustrate an exemplary distal end of the access device of FIG. 3 and an introducer.

FIGS. 6A, 6B, 6C, and 6D illustrate an exemplary patient interface.

### DETAILED DESCRIPTION

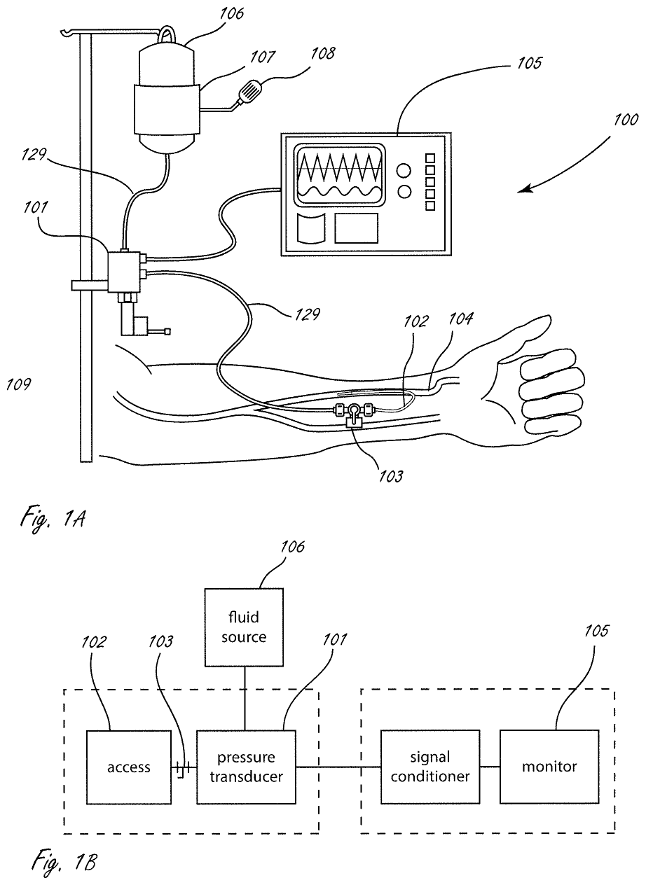

As indicated above, FIG. 1A illustrates the primary components of an exemplary traditional intravascular blood pressure monitoring system 100 configured for monitoring arterial pressure. The patient interface comprises an access device 102 partially positioned in a blood vessel 104 (e.g., a radial artery as shown), a valve (e.g., stopcock) 103, a pressure transducer 101, and a tubing set comprising a fluidic path 129 for fluidically coupling the described components. The system also comprises a fluid reservoir 106 of sterile fluid (e.g., saline) to constantly flush the transducer and associated lines, a pressurizing device 108 (e.g., a pressure bulb) adapted to apply pressure to the fluid reservoir 106 with pressure cuff 107, associated tubing to connect the fluid reservoir to the pressure transducer, and an instrument or monitor 105 to power and condition the signal from the pressure transducer and other transducers, monitor and record and/or present a representation of the signals detected by the transducers. The fluid is used to flush the transducer and thereby prevent blood from clotting within the fluidic path, which may cause the pressure detected at the pressure transducer to not be reflective of the actual physiological pressure. Such a system can also be adapted to access arterial blood for the purposes of collecting blood samples when they are required on a relatively frequent basis, such as when blood gas values are required. FIG. 1B illustrates a block diagram representation of such a system from FIG. 1A.

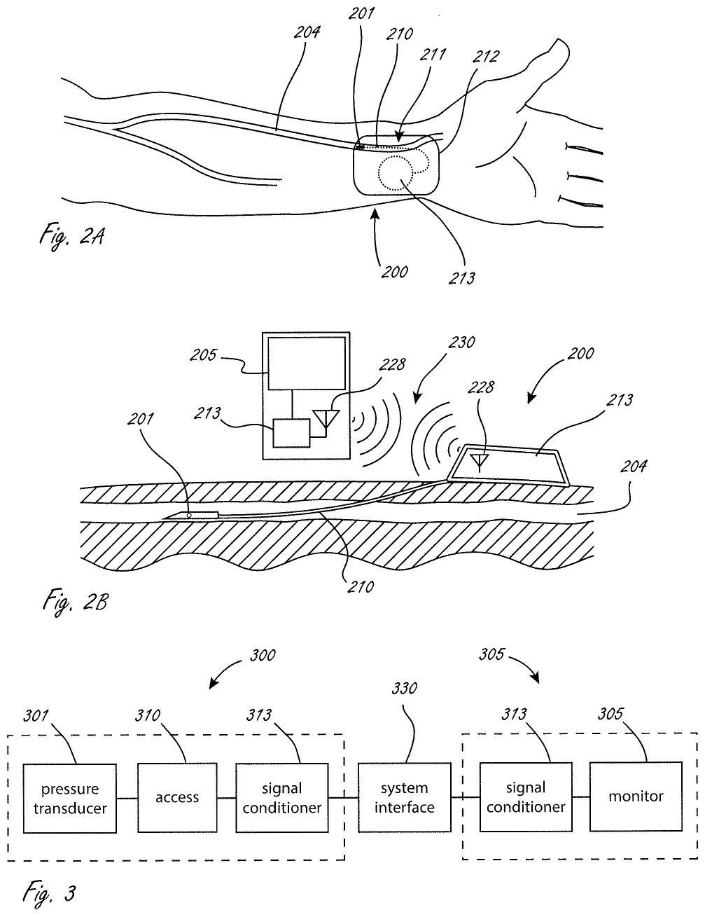

FIGS. 2A and 2B illustrate various views and/or aspects of some embodiments of the ARLIM systems herein. The system embodiment in FIGS. 2A and 2B includes: a patient interface 200 and a monitor 205. The patient interface 200 comprises an indwelling portion 211 and an external portion 212. The indwelling portion 211 comprises an access device 210 and a pressure transducer 201. The external portion comprises signal conditioning components 213 and a system interface 230 facilitated by RF transceivers 228 adapted to communicate between monitor 205 and patient interface 300. The monitor is capable of at least one of monitoring the data stream provided by the patient interface, processing the data, sending the data to another user, etc. FIG. 2A illustrates a patient interface 200 comprising an access device 210 that has been positioned in a radial artery 204. As illustrated, the pressure transducer 201 is disposed on a distal region (in this embodiment the distal end) of the access device. In other embodiments the access device provides a transmission path for pressure pulses to a pressure transducer disposed in the external portion of the access device.

FIG. 3 illustrates in a block diagram one embodiment of a patient interface shown generally in FIGS. 2A and 2B. In this exemplary system, the pressure transducer 301 is disposed at the distal end of the access device 310 of the indwelling portion 311 of the patient interface. The patient interface signal conditioning component 313 is powered and comprises an RF transceiver. In turn, the signal conditioner 313 in the monitor 305 comprises an RF transceiver and the system interface 330 is an RF link, such as a Bluetooth link. In such an embodiment, information may be transmitted from the patient interface component to the monitor component, such as, without limitation, calibration factors, changes in data collection rate, or characteristics of the pressure waveform, data precision.

FIG. 4 illustrates an exemplary embodiment of a system shown generally in FIGS. 2A and 2B, wherein the patient interface 400 comprises a pressure transducer 401 and a passive signal conditioner 413. As illustrated, the signal conditioning component 413 comprises an antenna 415 and additional circuit elements (not shown). In such a device, changes in the transducer characteristic associated with changing pressure cause a change in a characteristic as measured by the system interface, not shown. This change can be expressed as a change in impedance and/or resonant frequency of the patient interface. In such a system power is derived from the RF link.

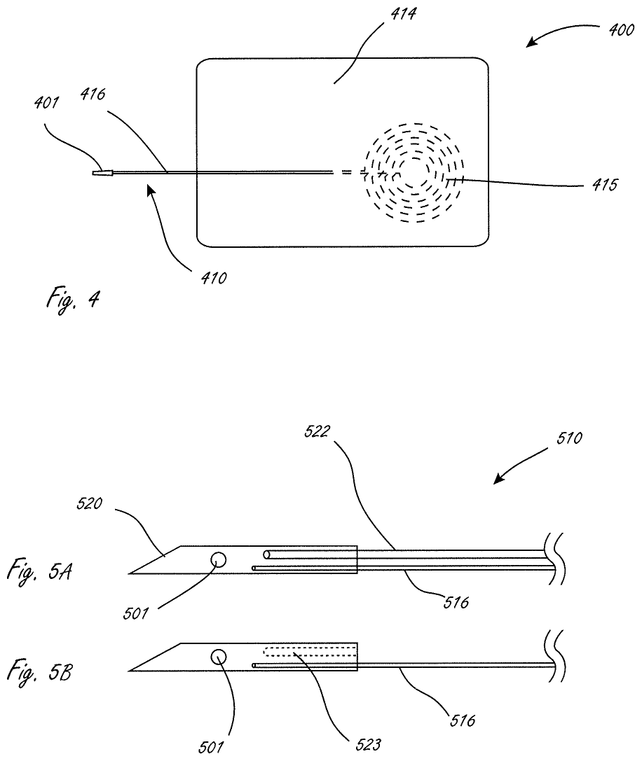

FIGS. 5A and 5B illustrates a distal region of an exemplary access component 510, which comprises a pressure transducer 501 disposed near the distal end. The distal end is configured with a sharpened end, or sharps component 520, for insertion through the artery wall. The sharps component is coupled to the signal conditioning component via an electrical interface such as a cable or flexible circuit 516. To provide the access device with sufficient rigidity to deliver the pressure transducer into the artery, the sharps component 520 includes an introducer interface 523 adapted to be releasably interfaced with an introducer, such as a stylet 522. Once delivered, the stylet may be removed, thereby providing a very small cross section and very flexible indwelling access component, which minimizes trauma to the patient. In some embodiments the introducer (e.g., stylet) and sharps component can also be non-releasably secured. The sharps component may also be referred to herein as a sharpened distal housing, which may be made of multiple components secured together to form the housing rather than a single housing structure made from a single starting material.

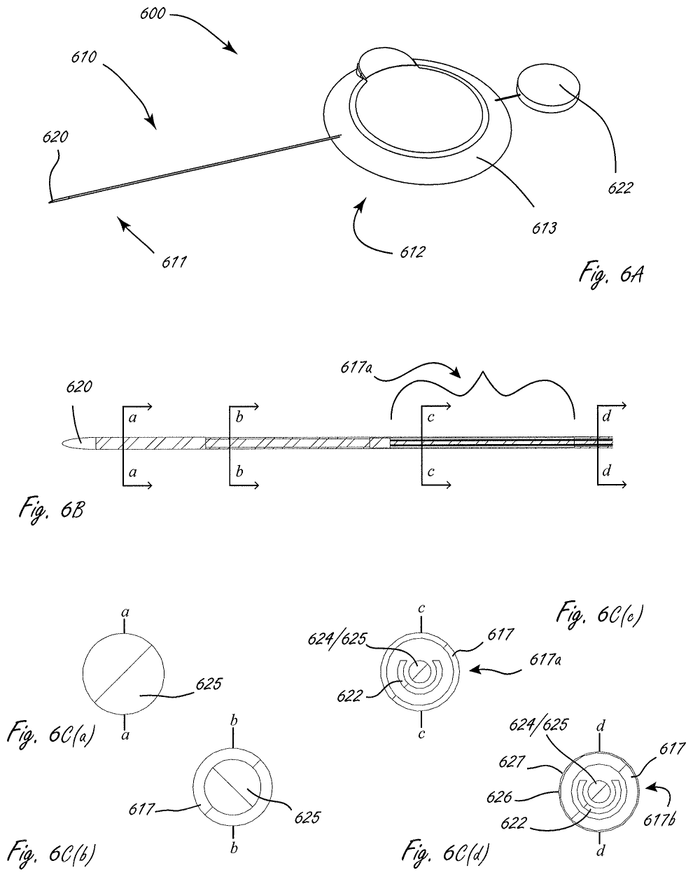

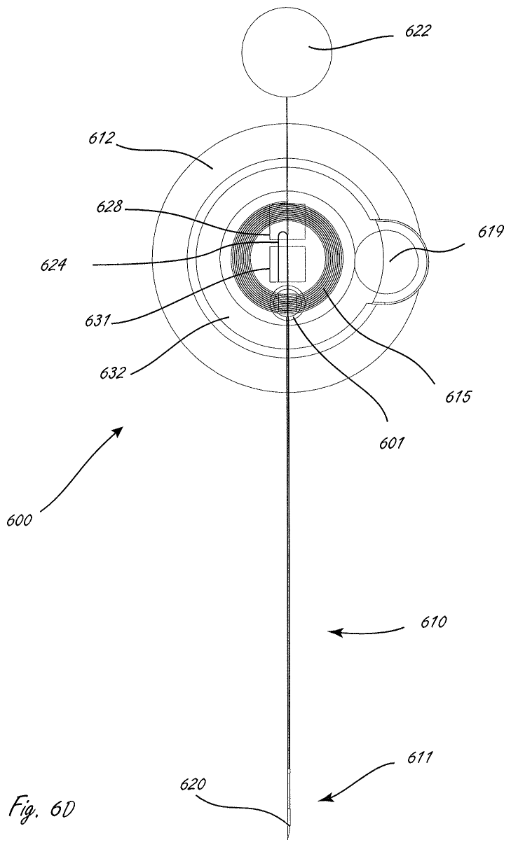

FIGS. 6A-6D (FIG. 6C is in fact four cross-sectional views from FIG. 6B) illustrate an exemplary embodiment of a patient interface device, in which the pressure transducer is housed in the external portion of the patient interface. The pressure transducer is operably coupled to blood pressure via a pressure coupling fluid (e.g. liquid) or gel within sealed access device 610. The device is thus adapted and configured to transmit pressure pulses from the blood to the pressure transducer. The access device 610 comprises a tubular outer member or catheter 617 typically constructed of an elastomeric material (silicone, poly urethane PEBAX, or other such material, or copolymers of such materials), and in some embodiments covered in areas with a thin, more rigid polymeric material (FEP, PTFE, PE, or other such materials or copolymers of such materials). The access device also includes an inner structure 625, and a removable delivery stylet 622. The tubular catheter member 617 interfaces at its distal end with a distal section of the inner structure 625 in an interference fit, as can be seen in FIG. 6B, and in the cross section b-b shown in FIG. 6C(b). As shown, tubular catheter member 617 is disposed around inner structure 625 in an interference fit with inner structure 625. The most distal portion of the inner structure 625 comprises a sharpened feature 620 to allow insertion of the access device into the vessel through tissues.

The distal region of the indwelling portion of the catheter is adapted to act as a one-way valve, which allows coupling media delivered under pressure from within the access device to be delivered to the blood, but does not allow blood to enter the inside of the catheter. As such, the blood is isolated from the pressure transmitting media but the fluid-pressure coupling region may be purged and/or filled with the pressure coupling media. In this particular embodiment, and in reference to section b-b, tubular member 617 is an elastomeric material, while inner member 625 is a much stiffer material such as (for example) stainless steel or a relatively hard plastic. When pressure transmitting media is delivered under pressure into the access device, the elastomeric material of tubular member 617 will distend slightly (relative to inner member 625) in response to the increase in fluid pressure, which will allow fluid to pass out of the catheter and into the blood. When the pressure transmitting media is no longer delivered, the pressure from the blood external to the catheter will maintain the interference fit between tubular member 617 and inner member 625, thus preventing blood from entering the indwelling portion. In this manner, the pressure coupling volume within the indwelling portion can be purged of air, and optionally fluids may be delivered to the blood stream

Pressure pulses are transmitted into the pressure transmitting media via a relatively thin-walled section 617a of the catheter portion of the access device, which is compliant enough to allow pressure pulses to be transmitted from the blood into the coupling pressure media. The blood is thus in pressure communication with the pressure transducer, but the blood is not in direct physical contact with the media inside the catheter. In alternate embodiments the section 617a may encompass less than 360 degrees of the tubular member.

FIG. 6A illustrates some exemplary features of a patient interface device 600. FIG. 6B is a side view of a distal region of indwelling access device 611, and shows four cross sections. FIG. 6C illustrates the four selected cross sections taken as shown in FIG. 6B. Exemplary aspects of the patient interface 600 are an access device 610 comprising an indwelling portion 611 and an external portion 612 for coupling pressure pulses to the signal conditioning unit 613, and an introducer, such as a delivery stylet 622, configured to stiffen access components structurally during the delivery process. The stylet passes through signal conditioning unit 613 during delivery of the access component and is removed after delivery.

FIG. 6C shows four cross sections taken from FIG. 6B. The distal end of the access device, which is distal to section A-A, comprises a sharpened end 620 and can be a solid section, such as a solid stainless steel section. Section B-B shows an interface between an outer, elastomeric, non-covered, section of catheter 617, and the inner structure 625 which comprises a one-way valve, allowing fluids delivered from the catheter to be released into the bloodstream and/or the fluid coupling region to be purged and or filled (as described above). The section C-C is within the catheter blood pressure capturing zone, and includes a thin walled section 617a of catheter wall 617, and a reduced cross section inner member or wire portion 624 that acts as a tether to prevent losing the distal end region and interface with the stylet during delivery. Section D-D is within the pressure transmitting zone and comprises the tether 624 or other elongate device, and a thicker walled 617b′ portion of the catheter 617. This section of the catheter may, in addition, be covered with a non-elastomeric polymer such as a poly ethylene or fluoropolymer to add additional stiffness to the catheter. The optionally removable stylet is shown in the sections C-C and D-D, configured to slideably interface with the tether or other elongate device. In alternative embodiments, only a portion of the wall in 6C(c) is thinner (i.e., less than 360 degrees), such as half of the wall, or a quarter of the wall.

FIG. 6D illustrates the patient interface 600 with some components contained within the signal conditioner 613 comprised within the external portion 612 made visible. As illustrated a battery 619 is used to power the signal conditioning component 613 comprising pressure transducer 601 and conditioning circuitry 631; conditioning circuitry 631 converts the pressure signal into a radio signal compatible with being broadcast by the RF transceiver 628 which interfaces with antenna 615. Typically, the pressure signal will be converted to a digital format and passed to the RF transceiver 628. Also shown is the proximal termination of the tether 624 anchored in the body of the signal conditioner housing 632. Without the stylet, only the distal region of the indwelling portion has sufficient stiffness to be advanced into and through the vessel. The stylet, when inserted, extends along more than 50% of the indwelling portion (and in this embodiment more than 75%), and allows the much more flexible proximal section to be advanced through the vessel.

### ADDITIONAL EXAMPLES

1. An intra-arterial blood pressure system, comprising a patient interface and a monitor, at least a portion of the patient interface sized to be disposed in an artery, such as a radial artery.

2. The system of claim 1 wherein the patient interface includes a pressure transducer, optional adapted to be inside the artery or disposed in an external component outside the artery.

3. The system of any claim herein wherein the monitor is a component in wireless communication with the patient interface.

4. The system of any claim herein wherein the patient interface includes an indwelling portion and an external portion.

5. The system of any claim herein wherein the patient interface includes an indwelling portion, which is adapted to be reversibly secured to a stiffening component to stiffen at least a portion of the indwelling portion during delivery, and cause the indwelling portion to be less stiff after its removal.

6. The system of any claim herein wherein a pressure transducer is disposed at a distal region, optionally a distal end, of an access device.

7. The system of any claim herein wherein a stiffening component is an elongate device, such as an introducer stylet.

8. The system of any claim herein wherein the distal end of an access device is sharped, to allow it to be pierced through a patient's skin.

9. The system of any claim herein wherein an access device of a patient interface includes a pressure capturing zone, adapted to transmit blood pressure to a pressure transducer.

10. The system of any claim herein wherein a pressure capture zone includes a relatively thin walled portion of an access device.

11. The system of any claim herein wherein a pressure capture zone includes a fluid or gel therein.

12. The system of any claim herein further including a removable introducer stylet.

13. The system of any claim herein wherein an external portion of a patient interface includes system conditioning components.

What is claimed is:

1. An intravascular blood pressure monitoring system, comprising:
a patient interface including
an indwelling portion that is adapted to be positioned within a blood vessel, the indwelling portion comprising a tubular member, at least a portion of the tubular member configured to transmit fluid pressure from blood that is external to the tubular member to a fluid disposed within the tubular member, the tubular member adapted to isolate the fluid from the blood, the fluid within the tubular member being in pressure communication with a pressure transducer, and
a stiffening member interface adapted to releasably interface with a stiffening member to stiffen at least a portion of the indwelling portion while delivering the indwelling portion to a position within the subject, and causing the indwelling portion to be less stiff after its removal.

2. The system of claim 1, wherein the pressure transducer is adapted to be disposed outside of the blood vessel.

3. The system of claim 2, wherein the patient interface comprises an external portion, the external portion configured to be secured to the skin of a subject.

4. The system of claim 1, wherein the outer wall has a thickness less than 0.25 mm.

5. The system of claim 1, wherein the indwelling portion has a section proximal to the elongate section, wherein a thickness of an outer wall in the proximal section is greater than the thickness of the outer wall in the elongate section.

6. The system of claim 5, wherein the fluid is disposed in a lumen through which the stiffening member is configured to be positioned.

7. The system of claim 6, further comprising a central elongate member secured to a section of the indwelling portion distal to the elongate section, the central elongate member configured with a surface to interface with the stiffening element.

8. The system of claim 1, the indwelling portion further comprising a one way valve distal to the elongate section, allowing fluid to pass only distally through the one way valve.

9. The system of claim 1, wherein the indwelling portion includes a sharpened distal end.

10. The system of claim 1, wherein the patient interface includes a communication component adapted to communicate with an external monitor.

11. An intravascular blood pressure monitoring apparatus, comprising:
a patient interface including
an indwelling portion that is adapted to be positioned within a blood vessel, the indwelling portion including a pressure transducer disposed in a distal region of the indwelling portion; and
a stiffening member interface adapted to releasably interface with a stiffening member to stiffen at least a portion of the indwelling portion while delivering the indwelling portion to a position within the subject, and causing the indwelling portion to be less stiff after its removal.

12. The apparatus of claim 11, wherein the pressure transducer is in electrical communication with a signal conditioner.

13. The apparatus of claim 11, wherein the indwelling portion has a sharpened distal end.

14. The apparatus of claim 11, wherein the patient interface includes a communication component adapted to communicate with an external monitor.

15. The apparatus of claim 11, wherein the patient interface comprises an external portion, the external portion configured to be secured to the skin of a subject.
